(19) United States
(12) Patent Application Publication
(10) Pub. No.: US 2023/0061869 A1
(43) Pub. Date: Mar. 2, 2023

(54) SYSTEM AND METHODS FOR BEAMFORMING SOUND SPEED SELECTION

(71) Applicant: GE Precision Healthcare LLC, Milwaukee, WI (US)

(72) Inventor: Kenneth W. Rigby, Clifton Park, NY (US)

(21) Appl. No.: 17/446,094

(22) Filed: Aug. 26, 2021

(51) Int. Cl. A61B 8/08; G01S 7/4865; G01S 7/52

(52) U.S. Cl. CPC A61B 8/5207; G01S 7/4865; G01S 7/52095; G01S 7/52036; G01S 7/52046

(57) ABSTRACT

Methods and systems are provided for receiving beamforming of ultrasound signals to generate ultrasound images with increased resolution. In one example, a method includes calculating a respective beamforming quality metric for each of a plurality of beamforming sound speeds, each beamforming quality metric calculated on ultrasound receive channel signals time-delayed based on a respective beamforming sound speed, identifying a target beamforming sound speed based on the beamforming quality metrics, and generating an ultrasound image using the target beamforming sound speed.

### TECHNICAL FIELD

Embodiments of the subject matter disclosed herein relate to ultrasound imaging, and more particularly, to improving image quality for ultrasound imaging.

### BACKGROUND

Medical ultrasound is an imaging modality that employs ultrasound waves to probe the internal structures of a body of a patient and produce a corresponding image. For example, an ultrasound probe comprising a plurality of transducer elements emits ultrasonic pulses which reflect or echo, refract, or are absorbed by structures in the body. The ultrasound probe then receives reflected echoes, which are processed into an image. Ultrasound images of the internal structures may be saved for later analysis by a clinician to aid in diagnosis and/or displayed on a display device in real time or near real time.

### SUMMARY

In an embodiment, a method includes calculating a respective beamforming quality metric for each of a plurality of beamforming sound speeds, each beamforming quality metric calculated using ultrasound receive channel signals time-delayed based on a respective beamforming sound speed, identifying a target beamforming sound speed based on the beamforming quality metrics, and generating an ultrasound image using the target beamforming sound speed. In some examples, the target beamforming sound speed may be used for calculating receive beamforming time delays, transmit beamforming time delays, or both receive and transmit beamforming time delays when generating the ultrasound image.

The above advantages and other advantages, and features of the present description will be readily apparent from the following Detailed Description when taken alone or in connection with the accompanying drawings. It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

Various aspects of this disclosure may be better understood upon reading the following detailed description and upon reference to the drawings in which:

FIG. 1 shows a block diagram of an exemplary embodiment of an ultrasound system;

FIG. 2 is a block diagram showing a receiver which forms part of the system of FIG. 1;

FIG. 3 is a diagram illustrating a calculation for an optimal beamforming sound speed based on a generated best fit to a polynomial for a plurality of data values representing beamforming quality at a plurality of beamforming sound speeds;

FIGS. 4A-4B show generated ultrasound images using a suboptimal beamforming sound speed and an optimal beamforming sound speed, respectively;

FIG. 5 is a diagram illustrating a plurality of calculations for optimal beamforming sound speeds over a plurality of scanning regions during an ultrasound scan;

FIG. 6 shows a generated ultrasound image using a closest beamforming sound speed to a calculated optimal beamforming sound speed for a plurality of regions that are denoted by region-of-interest markers;

FIG. 7 is a flow chart illustrating an example method for generating an ultrasound image based on an optimal beamforming sound speed calculation; and

FIG. 8 is a flow chart illustrating an example method for calculating beamforming quality metrics from a coherence factor.

### DETAILED DESCRIPTION

Medical ultrasound imaging typically includes the placement of an ultrasound probe including one or more transducer elements onto an imaging subject, such as a patient, at the location of a target anatomical feature (e.g., abdomen, chest, etc.). Images are acquired by the ultrasound probe and are displayed on a display device in real time or near real time (e.g., the images are displayed once the images are generated and without intentional delay). During a procedure when the ultrasound probe acquires an image, transmit beamforming and receive beamforming may be used. Time delays may be implemented during transmit beamforming and receive beamforming to alter an angle and focus range of beamforming when acquiring the ultrasound image. Beamforming time delays may be based on a speed that ultrasound echoes travel through an imaging medium (e.g., tissue). Such an assumed speed may typically be a default speed set by the system. Different imaging mediums (e.g., fatty tissue, liver tissue) exhibit different sound propagation speeds, meaning a speed at which ultrasound travels through a first medium may be different from a speed at which ultrasound travels through a second medium. As a result, a predetermined or default assumed speed set by the system may result in a loss of image resolution depending on the imaging medium or variance of imaging mediums covered during an ultrasound scan. Conventional ultrasound systems may allow an ultrasound operator to manually adjust the assumed sound speed used in calculating beamforming time delays, but various factors may inhibit proper selection of the sound speed (e.g., user inexperience, time constraints) which may result in ultrasound images exhibiting lower resolutions. In addition, a single assumed sound speed may not be optimal when the sound speed varies within the medium. Moreover, manufacturers of ultrasound systems find that a majority of operators dislike having to make manual adjustments on an ultrasound system to optimize an image quality.

Thus, according to embodiments disclosed herein, an optimal beamforming sound speed may be automatically selected such that a generated ultrasound image during a scan using the optimal beamforming sound speed may exhibit an improvement to image resolution. Ultrasound data received by the transducers of the ultrasound probe prior to processing into an image (referred to herein as channel data) may be analyzed to calculate and identify beamforming sound speeds such that an optimal beamforming sound speed may be selected before generating ultrasound images displayed to the operator, and then ultrasound images may be generated after the optimal beamforming sound speed is selected. A beamforming quality metric may be calculated with the channel data, allowing for an automatic selection of ultrasound imaging parameters, such as a beamforming sound speed used for transmit beamforming calculations, receive beamforming calculations, or both transmit and receive beamforming calculations, which may be adjusted in order to increase ultrasound image resolution. The beamforming quality metric may be calculated from a coherence factor, which may be a ratio of an amplitude of receive signals summed coherently and an amplitude of receive signals summed incoherently, which may be calculated from channel data and which may act as an indicator of a possible ultrasound image resolution. An ultrasound image generated from automatically selected ultrasound imaging parameters may exhibit higher image resolutions without operator intervention.

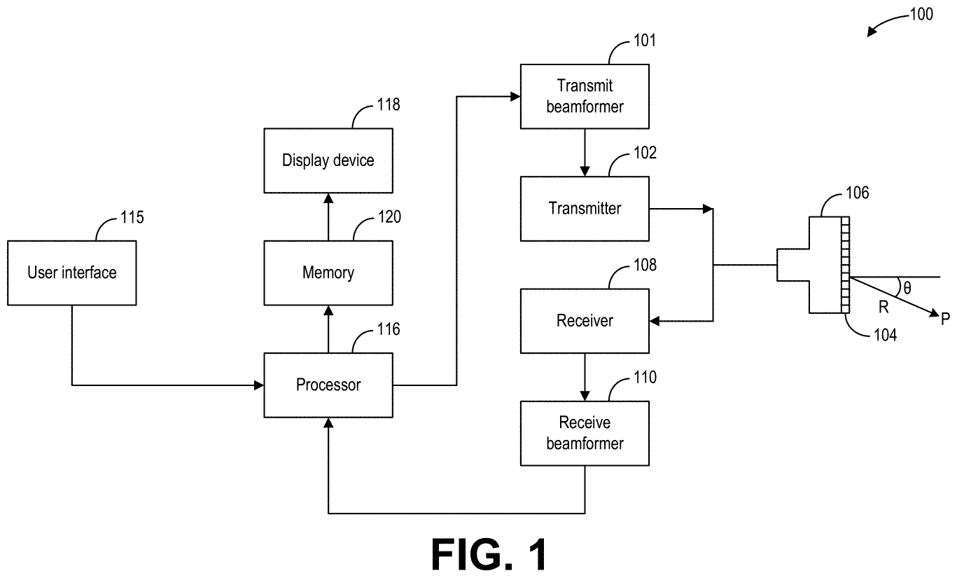

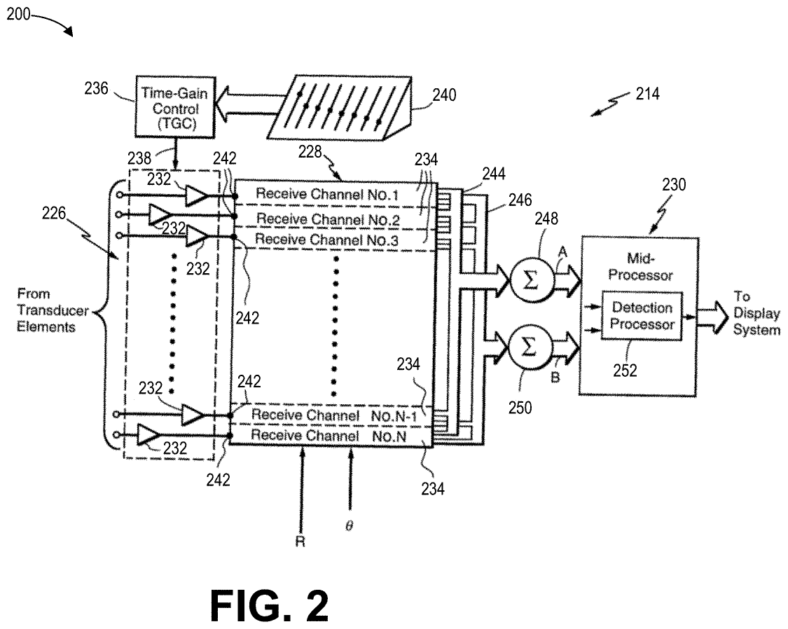

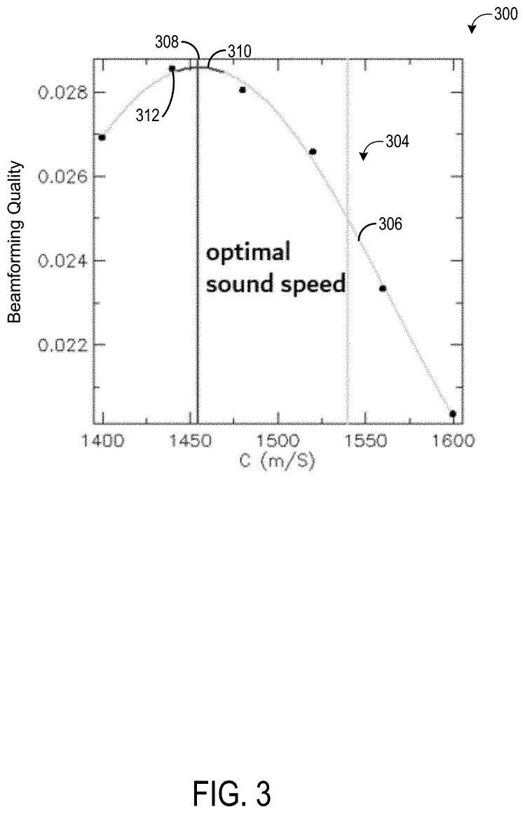

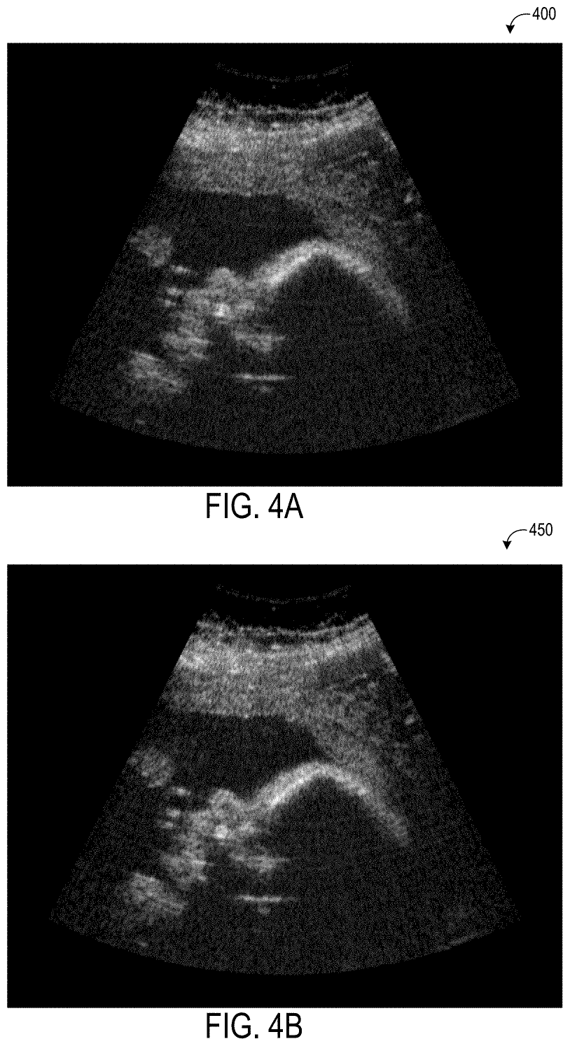

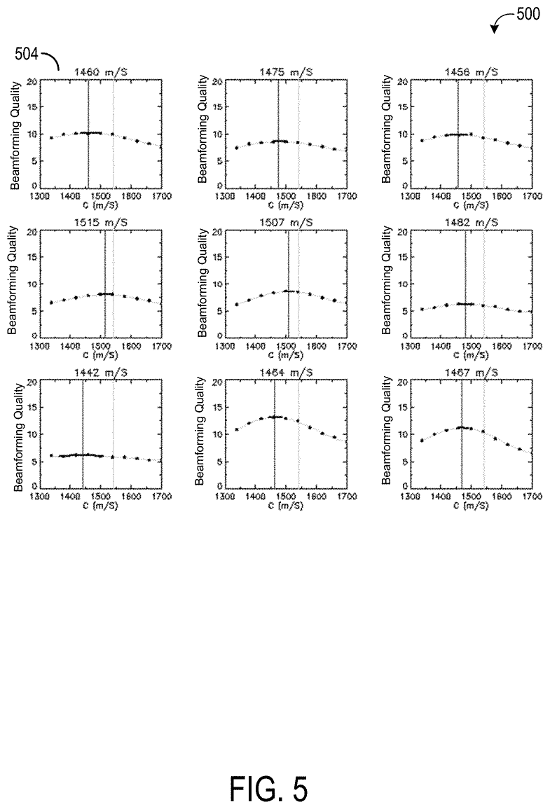

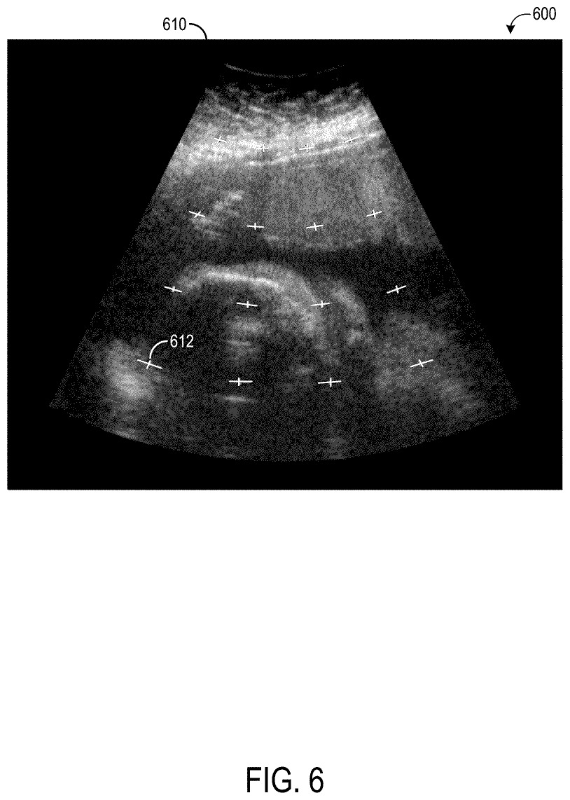

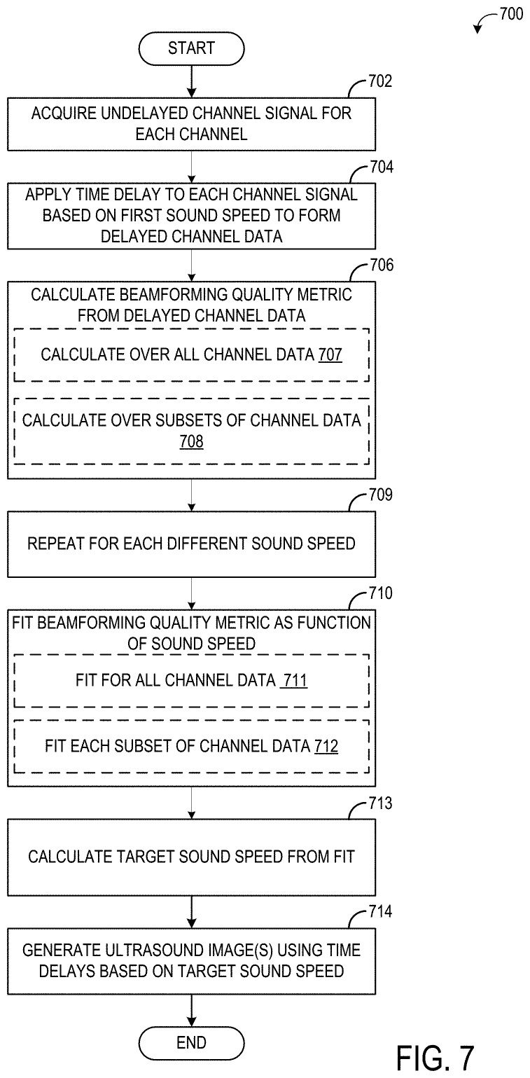

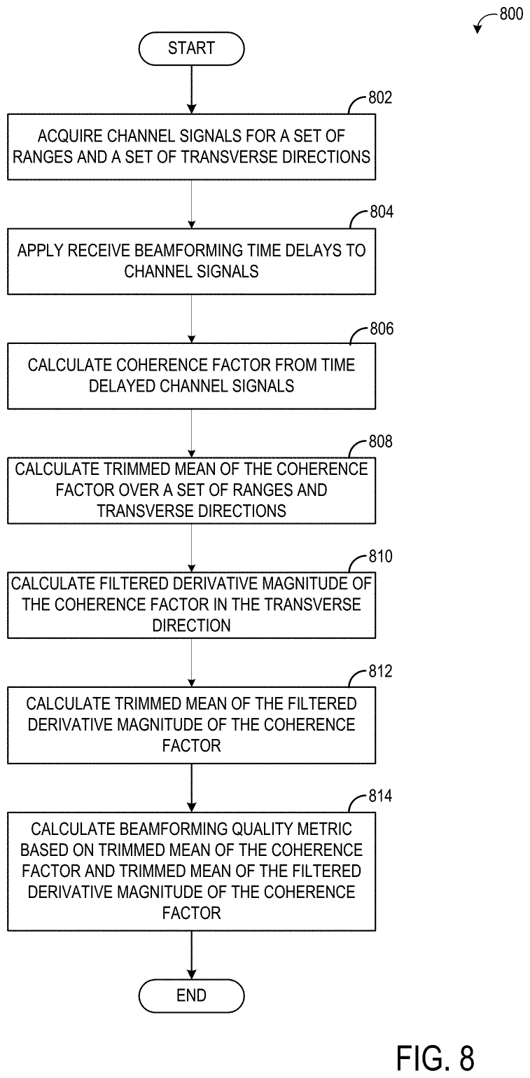

An example ultrasound system including an ultrasound probe, a display device, and an imaging processing system are shown in FIG. 1. Via the ultrasound probe, ultrasound data may be acquired and ultrasound images may be calculated and displayed on the display device. A receiver of the system of FIG. 1 with multiple receive channels for receive beamforming is shown in FIG. 2. A graph illustrating a method for calculating an optimal beamforming sound speed using data obtained via the multiple receive channels is shown in FIG. 3. An ultrasound image generated from a suboptimal beamforming sound speed is shown in FIG. 4A. An ultrasound image generated from an optimal beamforming sound speed selected according to the graph of FIG. 3 is shown in FIG. 4B. A plurality of graphs illustrating a method for calculating optimal beamforming sound speeds over subdivided regions of an ultrasound scanning region is shown in FIG. 5. An ultrasound image that may be generated using optimal beamforming sound speeds for each subdivided region in a scanning region is shown in FIG. 6. An optimal beamforming sound speed may be calculated and used to generate an ultrasound image according to the method of FIG. 7, where the optimal beamforming sound speed is selected based on a coherence factor that is calculated according to the method of FIG. 8.

Referring to FIG. 1, a schematic diagram of an ultrasound imaging system 100 in accordance with an embodiment of the disclosure is shown. The ultrasound imaging system 100 includes a transmit beamformer 101 and a transmitter 102 that drives elements (e.g., transducer elements) 104 within a transducer array, herein referred to as probe 106, to emit pulsed ultrasonic signals (referred to herein as transmit pulses) into a body (not shown). According to an embodiment, the probe 106 may be a one-dimensional transducer array probe. However, in some embodiments, the probe 106 may be a two-dimensional matrix transducer array probe. As explained further below, the transducer elements 104 may be comprised of a piezoelectric material. When a voltage is applied to a piezoelectric crystal, the crystal physically expands and contracts, emitting an ultrasonic wave. In this way, transducer elements 104 may convert electronic transmit signals into acoustic transmit beams.

After the elements 104 of the probe 106 emit pulsed ultrasonic signals into a body (of a patient), the pulsed ultrasonic signals reflect from structures within an interior of the body, like blood cells or muscular tissue, to produce echoes that return to the elements 104. The echoes are converted into electrical signals, or ultrasound data, by the elements 104 and the electrical signals are received by a receiver 108. The electrical signals representing the received echoes are passed through a receive beamformer 110 that outputs ultrasound data.

The echo signals produced by transmit operation reflect from structures located at successive ranges along the transmitted ultrasonic beam. The echo signals are sensed separately by each transducer element and a sample of the echo signal magnitude at a particular point in time represents the amount of reflection occurring at a specific range. Due to the differences in the propagation paths between a reflecting point P and each element, however, these echo signals are not detected simultaneously. Receiver 108 amplifies the separate echo signals, imparts a calculated receive time delay to each, and sums them to provide a single echo signal which approximately indicates the total ultrasonic energy reflected from point P located at range R along the ultrasonic beam oriented at the angle θ.

The time delay of each receive channel continuously changes during reception of the echo to provide dynamic focusing of the received beam at the range R from which the echo signal is assumed to emanate based on an assumed sound speed for the medium.

Under direction of processor 116, the receiver 108 provides time delays during the scan such that steering of receiver 108 tracks the direction θ of the beam steered by the transmitter and samples the echo signals at a succession of ranges R so as to provide the time delays and phase shifts to dynamically focus at points P along the beam. Thus, each emission of an ultrasonic pulse waveform results in acquisition of a series of data points which represent the amount of reflected sound from a corresponding series of points P located along the ultrasonic beam.

According to some embodiments, the probe 106 may contain electronic circuitry to do all or part of the transmit beamforming and/or the receive beamforming. For example, all or part of the transmit beamformer 101, the transmitter 102, the receiver 108, and the receive beamformer 110 may be situated within the probe 106. The terms “scan” or “scanning” may also be used in this disclosure to refer to acquiring data through the process of transmitting and receiving ultrasonic signals. The term “data” may be used in this disclosure to refer to either one or more datasets acquired with an ultrasound imaging system. A user interface 115 may be used to control operation of the ultrasound imaging system 100, including to control the input of patient data (e.g., patient medical history), to change a scanning or display parameter, to initiate a probe repolarization sequence, and the like. The user interface 115 may include one or more of the following: a rotary element, a mouse, a keyboard, a trackball, hard keys linked to specific actions, soft keys that may be configured to control different functions, and a graphical user interface displayed on a display device 118.

The ultrasound imaging system 100 also includes a processor 116 to control the transmit beamformer 101, the transmitter 102, the receiver 108, and the receive beamformer 110. The processor 116 is in electronic communication (e.g., communicatively connected) with the probe 106. For purposes of this disclosure, the term “electronic communication” may be defined to include both wired and wireless communications. The processor 116 may control the probe 106 to acquire data according to instructions stored on a memory of the processor, and/or memory 120. The processor 116 controls which of the elements 104 are active and the shape of a beam emitted from the probe 106. The processor 116 is also in electronic communication with the display device 118, and the processor 116 may process the data (e.g., ultrasound data) into images for display on the display device 118. The processor 116 may include a central processor (CPU), according to an embodiment. According to other embodiments, the processor 116 may include other electronic components capable of carrying out processing functions, such as a digital signal processor, a field-programmable gate array (FPGA), or a graphic board. According to other embodiments, the processor 116 may include multiple electronic components capable of carrying out processing functions. For example, the processor 116 may include two or more electronic components selected from a list of electronic components including: a central processor, a digital signal processor, a field-programmable gate array, and a graphic board. According to another embodiment, the processor 116 may also include a complex demodulator (not shown) that demodulates the real RF (radio-frequency) data and generates complex data. In another embodiment, the demodulation can be carried out earlier in the processing chain. The processor 116 is adapted to perform one or more processing operations according to a plurality of selectable ultrasound modalities on the data. In one example, the data may be processed in real-time during a scanning session as the echo signals are received by receiver 108 and transmitted to processor 116. For the purposes of this disclosure, the term “real-time” is defined to include a procedure that is performed without any intentional delay. For example, an embodiment may acquire images at a real-time rate of 7-20 frames/sec. The ultrasound imaging system 100 may acquire 2D data of one or more planes at a significantly faster rate. However, it should be understood that the real-time frame-rate may be dependent on the length of time that it takes to acquire each frame of data for display. Accordingly, when acquiring a relatively large amount of data, the real-time frame-rate may be slower. Thus, some embodiments may have real-time frame-rates that are considerably faster than 20 frames/sec while other embodiments may have real-time frame-rates slower than 7 frames/sec. The data may be stored temporarily in a buffer (not shown) during a scanning session and processed in less than real-time in a live or off-line operation. Some embodiments of the invention may include multiple processors (not shown) to handle the processing tasks that are handled by processor 116 according to the exemplary embodiment described hereinabove. For example, a first processor may be utilized to demodulate and decimate the RF signal while a second processor may be used to further process the data, for example by augmenting the data as described further herein, prior to displaying an image. It should be appreciated that other embodiments may use a different arrangement of processors.

The ultrasound imaging system 100 may continuously acquire data at a frame-rate of, for example, 10 Hz to 30 Hz (e.g., 10 to 30 frames per second). Images generated from the data may be refreshed at a similar frame-rate on display device 118. Other embodiments may acquire and display data at different rates. For example, some embodiments may acquire data at a frame-rate of less than 10 Hz or greater than 30 Hz depending on the size of the frame and the intended application. A memory 120 is included for storing processed frames of acquired data. In an exemplary embodiment, the memory 120 is of sufficient capacity to store at least several seconds' worth of frames of ultrasound data. The frames of data are stored in a manner to facilitate retrieval thereof according to its order or time of acquisition. The memory 120 may comprise any known data storage medium.

In various embodiments of the present invention, data may be processed in different mode-related modules by the processor 116 (e.g., B-mode, Color Doppler, M-mode, Color M-mode, spectral Doppler, Elastography, TVI, strain, strain rate, and the like) to form 2D or 3D data. For example, one or more modules may generate B-mode, color Doppler, M-mode, color M-mode, spectral Doppler, Elastography, TVI, strain, strain rate, and combinations thereof, and the like. As one example, the one or more modules may process color Doppler data, which may include traditional color flow Doppler, power Doppler, HD flow, and the like. The image lines and/or frames are stored in memory and may include timing information indicating a time at which the image lines and/or frames were stored in memory. The modules may include, for example, a scan conversion module to perform scan conversion operations to convert the acquired images from beam space coordinates to display space coordinates. A video processor module may be provided that reads the acquired images from a memory and displays an image in real time while a procedure (e.g., ultrasound imaging) is being performed on a patient. The video processor module may include a separate image memory, and the ultrasound images may be written to the image memory in order to be read and displayed by display device 118.

In various embodiments of the present disclosure, one or more components of ultrasound imaging system 100 may be included in a portable, handheld ultrasound imaging device. For example, display device 118 and user interface 115 may be integrated into an exterior surface of the handheld ultrasound imaging device, which may further contain processor 116 and memory 120. Probe 106 may comprise a handheld probe in electronic communication with the handheld ultrasound imaging device to collect raw ultrasound data. Transmit beamformer 101, transmitter 102, receiver 108, and receive beamformer 110 may be included in the same or different portions of the ultrasound imaging system 100. For example, transmit beamformer 101, transmitter 102, receiver 108, and receive beamformer 110 may be included in the handheld ultrasound imaging device, the probe, and combinations thereof.

After performing a two-dimensional ultrasound scan, a block of data comprising scan lines and their samples is generated. After back-end filters are applied, a process known as scan conversion is performed to transform the two-dimensional data block into a displayable bitmap image with additional scan information such as depths, angles of each scan line, and so on. During scan conversion, an interpolation technique is applied to fill missing holes (i.e., pixels) in the resulting image. These missing pixels occur because each element of the two-dimensional block should typically cover many pixels in the resulting image. For example, in current ultrasound imaging systems, a bicubic interpolation is applied which leverages neighboring elements of the two-dimensional block. As a result, if the two-dimensional block is relatively small in comparison to the size of the bitmap image, the scan-converted image will include areas of less than optimal or low resolution, especially for areas of greater depth.

Turning now to FIG. 2, it shows an embodiment 200 of a receiver 214 comprising three sections, including a time-gain control section 226, a receive beamforming section 228, and a mid-processor 230. In one example, receiver 214 may be a non-limiting example of receiver 108 of FIG. 1. The time-gain control (TGC) section 226 includes a respective amplifier 232 for each of the receive channels 234, and a time-gain control circuit 236 is provided for controlling gain of amplifiers 232. The input of each amplifier 232 is coupled to a respective one of transducer elements 104 to amplify an echo signal which it receives. The amount of amplification provided by amplifiers 232 is controlled through a control line 238 driven by TGC circuit 236, the latter being set by hand operation of potentiometers 240.

The receive beamforming section 228 of receiver 214 includes a plurality of receive channels 234, each receive channel 234 receiving the analog echo signal from a respective amplifier 232 at a respective input 242. The analog signals are digitized and produced as a stream of signed digitized samples. These samples are respectively delayed in the receive channels such that when they are summed with samples from each of the other receive channels, the amplitude of the summed signals is a measure of the strength of the echo signal reflected from a point P located at range R on the steered beam e.

To properly sum electrical signals produced by the echoes impinging on each transducer element 104, time delays are introduced into each separate channel 234 of receiver 214. The time delay that is added to each receive channel may be based on an assumed speed of sound through the imaged tissue. In one example, the imaged tissue may include more fat than muscle in certain regions, affecting the time delay that may be applied when receive beamforming over that region as a result of a difference in propagation speed when ultrasound passes through fat and when ultrasound passes through muscle. Each receive channel 234 supplies, in addition to the delayed signed samples, the amplitude, or absolute value, of the delayed signed samples. The delayed signed samples are provided to a coherent summation bus 244, while the amplitudes of the delayed signed samples are provided to an incoherent summation bus 246. Coherent summation bus 244 sums the delayed signed samples from each receive channel 234 using pipeline summers 248 to produce a coherent sum (e.g., coherent sum A in FIG. 2). Incoherent summation bus 246 sums the amplitudes of the delayed signed samples from each receive channel 234 using pipeline summers 250 to produce an incoherent sum (e.g., incoherent sum B in FIG. 2).

Receiver mid-processor section 230 receives the coherently summed beam samples from pipeline summers 248 and receives the incoherently summed beam samples from pipeline summers 250. Mid-processor section 230 comprises a detection processor 252.

Detection processor 252 calculates and applies a coherence factor in accordance with the present disclosure. The coherence factor is calculated for each data sample and may be defined (at least in one example) to be the ratio of two quantities: the amplitude of the sum of the receive signals and the sum of the amplitudes of the receive signals. The ratio is calculated in detection processor 252 by calculating the absolute value of the coherent sum from pipeline summers 248 and then calculating the ratio of the absolute value of the coherent sum from pipeline summers 248 to the incoherent sum from pipeline summers 250. If the incoherent sum is zero, the ratio may be set to zero. In one example, the ratio is calculated by dividing the absolute value of the coherent sum by the incoherent sum and a small positive value, which avoids illegally dividing by zero when the incoherent sum is zero. In an example, the detection processor 252 is a non-limiting example of processor 116 of FIG. 1. In another example, the detection processor 252 is separate from, but operably coupled to, processor 116 of FIG. 1.

For the case of a RF beamformer, the signal from each channel is a real, signed quantity and the coherent sum is the sum of the signals. The incoherent sum is the sum of the absolute value of each signal (e.g., a sum of non-negative numbers). For the case of a baseband beamformer, the real signal from each channel may be demodulated to form a complex quantity, so that the coherent sum is also a complex quantity. The absolute value of the coherent sum may be a non-negative real quantity. The incoherent sum may be the sum of the absolute values of the channel signal; it may be a non-negative real quantity. In both cases, the ratio of the absolute value of the coherent sum to the incoherent sum may be a non-negative real quantity.

During an ultrasound scan, a transmitter may drive a probe such that ultrasonic energy produced may be directed in a beam. To accomplish this, the transmitter may impart a time delay to pulsed waveforms that may be applied to successive transducer elements in the probe. By adjusting time delays in a conventional manner, the beam of ultrasonic energy may be directed away from an axis perpendicular to the probe face by some angle, focusing the beam at a fixed range. By successively changing the time delays, an ultrasound scan may be performed as a result of the angle at which the beam is directed progressively changing.

A receive ultrasound beamformer may be used as part of an ultrasound imaging system. The ultrasound imaging system may use a transmit event from a transmit beamformer and a receive event from the receive beamformer to generate one line of an ultrasound image. The transmit beamformer may focus at one location of a scanning region, and the receive beamformer may focus at the same location. During receive beamforming, time delays may be associated with a propagation of ultrasound waves in relation to an angle and a time, based on an assumed sound propagation speed, at which an ultrasound wave is received by the receive beamformer.

Due to differing sound propagation speeds of anatomical mediums in an anatomical region, a speed at which an ultrasound wave propagates during an ultrasound scan may vary. A default propagation speed may be considered for the ultrasound scan depending on the anatomical region the probe may be transmitting over, which may be prone to error, resulting in lower image resolutions. Errors from using a default propagation speed during an ultrasound scan may occur in scanning regions that include muscle, fat, and bone, as a result of ultrasound waves propagating at different speeds through all mediums. When a receive beamformer operates with a default propagation speed that differs from an actual propagation speed, data received may be calculated erroneously, resulting in inaccurate imaging during a generation of an ultrasound image. In addition, when a transmit beamformer operates with a default propagation speed that differs from an actual propagation speed, the transmit focus may be degraded. In one example, an ultrasound scan may occur over a region including tissues which may have differences in their associated sound propagation speed, and an ultrasound image generated from the ultrasound scan using a default sound speed may have suboptimal image quality. Automatically selecting an optimal beamforming sound speed may result in improved image quality.

Turning now to FIG. 3, it shows an example of a graph 300 illustrating a calculated optimal beamforming sound speed. Graph 300 plots a beamforming quality metric as a function of a beamforming sound speed for a plurality of beamforming sound speeds. In one example, the beamforming quality may be calculated from a measure of the coherence of the receive signals, such as the coherence ratio, calculated in mid-processor 230 in FIG. 2 as a ratio of an amplitude of receive signals summed coherently and an amplitude of receive signals summed incoherently. In one example, six instances of a beamforming quality metric (e.g., at six different sound speeds) may be calculated. In an alternate embodiment of FIG. 3, fewer or more than six instances of a beamforming quality matric may be calculated. The beamforming quality metric at each beamforming sound speed may be represented by a plurality of data points 304, shown in graph 300.

In the example shown, an ultrasound scan is performed such that echoes are received from a subject following transmission of ultrasound signals via an ultrasound probe. The echoes are received by the ultrasound transducers of the ultrasound probe and the output of each transducer is sent along the channels for processing, as described above. The channel signals are then sequentially processed with different time delays based on a plurality of different sound speeds, herein 1400 m/s, 1440 m/s, 1480 m/s, 1520 m/s, 1560 m/s, and 1600 m/s, and the beamforming quality metric is calculated on the receive channel data for each sound speed. Thus, each data point represents the beamforming quality metric for a given beamforming sound speed. In this example, the beamforming sound speed for the transmit time-delay calculations is set to the default beamforming sound speed. The same channel data can be time-delayed using each desired receive beamforming sound speed. In an alternate example, the beamforming sound speed for the transmit time-delay calculations is set to the receive beamforming sound speed. In this case, a separate transmit for each desired sound speed is required and the beamforming quality metric is calculated using different channel data for each sound speed.

A best fit curve 306 may be illustrated on graph 302, representing an approximate relationship between the beamforming quality as a function of beamforming sound speed. Best fit curve 306 may be a polynomial with fewer coefficients than the number of beamforming sounds speeds. In one example, best fit curve 306 for six beamforming sound speeds may be a polynomial of degree 4 with its coefficients determined using standard methods. When fitting to polynomials, the beamforming sound speeds may be scaled to the range [−1,1] using the formula x=(2*c-cₘₐₓ-cₘᵢₙ), where x is the scaled sound speed, cₘₐₓ is the maximum beamforming sound speed and cₘᵢₙ is the minimum beamforming sound speed.

Using best fit curve 306, an optimal sound speed 308 and a range of uncertainty 310 may be calculated and determined from an equation associated with best fit curve 306. Optimal sound speed 308 may represent a beamforming sound speed at which a highest beamforming quality value is identified from best fit curve 306. The range of uncertainty 310 may represent a range of beamforming sound speeds in an increasing and decreasing direction from optimal sound speed 308 including optimal sound speed 308. In one example, range of uncertainty 310 may be a determined range that ends on either side of optimal sound speed 308 when a maximum absolute value of difference between optimal sound speed 308 and another beamforming sound speed is reached, with the maximum absolute difference Δf equal to the standard deviation of the beamforming quality values from the best fit curve. In another example, range of uncertainty 310 may be determined based on a second derivative of best fit curve 306 evaluated at the optimal sound speed 308. Near the optimal scaled sound speed x₀ 308, the best fit curve f(x) for the scaled sound speed can be approximated by an equation f(x)=(½)α(x-x₀)², where a is the second derivative of the best fit curve evaluated at the optimal scaled beamforming sound speed x₀. With this approximation, the lower and upper limits of the range of uncertainty of the scaled beamforming sound speed are given by an expression x₀±(2Δf/α)^{1/2}.

Once optimal sound speed 308 and range of uncertainty 310 are calculated and determined, a beamforming sound speed may be automatically selected for generating an ultrasound image. The range of uncertainty 310 may be used to automatically decide whether the optimal sound speed or the default sound speed should be used to generate the image presented to the operator. In one example, if the range of uncertainty 310 is a substantial fraction, in one example 50%, of the range of beamforming sound speeds tested, then the default beamforming sound speed should be used. The range of uncertainty 310 may be large (e.g., 50% of the range of speeds) when the best fit curve is a less than optimal approximation to the beamforming quality values, in which case the optimal sound speed calculated from the best fit curve may not result in an image better than the image calculated using the default sound speed. The range of uncertainty 310 may also be large when the beamforming quality values vary little with sound speed or when the fitted curve does not have a well-defined local maximum. In this case, the image calculated using the default beamforming sound speed should be presented to the operator. If the range of uncertainty 310 is not large, then, in one example, optimal sound speed 308 may be selected to generate an ultrasound image. In another example, a beamforming sound speed of a closest beamforming data point 312 to optimal sound speed 308 may be selected to generate an ultrasound image.

FIG. 4A shows an example of a first generated ultrasound image 400. In one example, first generated ultrasound image 400 may be a generated ultrasound image using a beamforming sound speed on a data point in FIG. 3 further away from a calculated optimal sound speed. FIG. 4B shows an example of a second generated ultrasound image 450. The images shown in FIGS. 4A and 4B are generated from the same ultrasound receive channel signals, but with different time-delays determined from different sound speeds. In one example, second generated ultrasound image 450 may be a generated ultrasound image using a beamforming sound speed that is closest to a calculated optimal sound speed. As a result, first generated ultrasound image 400 may have a lower beamforming quality metric than second generated ultrasound image 450, indicating lower image resolution and contrast.

Thus, by selecting a target beamforming sound speed that is closest to the beamforming sound speed that results in a highest beamforming quality metric for all ultrasound data for generating an image, image quality may be improved over conventional methods that may rely on a default beamforming sound speed. However, to further improve image quality, ultrasound data obtained to generate an image may be subdivided into regions and a target beamforming sound speed may be selected for each region, as described in more detail below, which may allow for more targeted beamforming sound speeds, particularly when imaging anatomy with varying sound propagation speeds.

Turning now to FIG. 5, it shows a plurality of graphs 500 illustrating calculated optimal beamforming sound speeds for a maximum beamforming quality over a subset of regions in an ultrasound scan. Each graph in the plurality of graphs 500, such a first graph 504, may be generated similarly to graph 302 of FIG. 3 by way of calculating best fit curves, calculating optimal sound speeds, calculating ranges of uncertainty, and selecting beamforming sound speeds for the region each graph represents. In one example, an ultrasound scanning region (which may include ultrasound data for generating an image) may be divided into nine regions, and a graph may be generated for each region. In an alternate embodiment of FIG. 5, an ultrasound scanning region may be divided into fewer or more than nine regions. Each graph in the plurality of graphs 500 may illustrate a plurality of beamforming data points with beamforming sound speed on the x-axis and beamforming quality on the y-axis. In one example, ten sound speeds may be used to calculate ten respective beamforming quality metrics and plotted at individual beamforming sound speeds for each region in the ultrasound scan. In an alternate embodiment of FIG. 5, fewer or more than ten instances of beamforming may be recorded and plotted. During each instance of beamforming, beamforming quality metrics may be calculated from channel data as it relates to each subset region such that each instance may be plotted as a data point based on the quantifiable beamforming quality metric calculated from channel data.

Dividing an ultrasound scanning region into a plurality of regions may result in different optimal sound speeds being calculated depending on the anatomical features in each region. In one example, a graph in the plurality of graphs 504 may represent a region including more fatty tissue than another graph in the plurality of graphs 504 representing a different region which may include less fatty tissue, resulting in different optimal beamforming sound speeds because of the different sound speeds of each region. In addition, since the propagation time for sound between a desired focus position and a transducer element is a function of the sound speed along the acoustic path between the desired focus position and the element position, the optimal beamforming sound speed for a desired focus position may not be the actual sound speed at that focus position. Dividing an ultrasound scanning region into a plurality of regions may lead to a generated ultrasound image with a higher beamforming quality than a generated ultrasound image using a singular beamforming sound speed for the whole ultrasound scanning region.

FIG. 6 shows an example of a generated ultrasound image 600 that may be generated using a plurality of calculated optimal beamforming sound speeds over individual regions as explained above with respect to FIG. 5. The regions may be chosen automatically or by the operator. A plurality of partition markers, such as partition marker 612, may be visually present to indicate where each region is located in ultrasound image 600. Dividing an ultrasound image, such as ultrasound image 610, into regions during an image generation may result in a higher image resolution as a result of selecting optimal beamforming data in each region.

Turning now to FIG. 7, it shows a flow chart illustrating an example method 700 for generating an ultrasound image using a target beamforming sound speed selected based on a beamforming quality metric to generate an ultrasound image with enhanced image resolution and/or contrast. Method 700 is described with regard to the systems and components of FIGS. 1-2, though it should be appreciated that the method 700 may be implemented with other systems and components without departing from the scope of the present disclosure. Method 700 may be carried out according to instructions stored in non-transitory memory of a computing device, such as memory 120, and executed by a processor of the computing device, such as processor 116. In some examples, method 700 may be carried out each time ultrasound data is acquired by the ultrasound probe and an image is formed from the ultrasound data, such that the frequency that method 700 is executed matches the frame rate of the ultrasound imaging. In other examples, method 700 may be carried out a predetermined frequency during ultrasound imaging that is less than the frame rate (e.g., once per second) and/or in response to a determination that the ultrasound probe has been moved or that the anatomy being imaged has changed.

At 702, method 700 includes acquiring an undelayed channel signal for each element in an ultrasound probe, such as the receive channels 234 as illustrated and described with respect to FIG. 2. Channel data may be acquired through receive beamforming before generating an ultrasound image. As explained above with respect to FIGS. 1 and 2, the channel signals include echoes detected by the ultrasound transducer elements, which are received following transmission of ultrasound signals from the ultrasound transducer elements of the ultrasound probe. In some examples, the channel signals sufficient for generating an entire ultrasound image may be acquired and saved in memory.

At 704, method 700 includes applying a respective time delay to each channel signal based on a first sound speed to form delayed channel data for each channel. The first sound speed may be a first sound speed of a set of possible sound speeds. The set of possible sound speeds may be predetermined or set by a user. In an example, the set of possible sound speeds may include three, four, five, six, or more sound speeds within a range of reasonable sound speeds based on the imaging task (e.g., imaging human anatomy may result in a range of 1400 m/s-1600 m/s). Applying time delays may include altering channel data based on assumed properties of an imaging medium an ultrasound wave propagates through. As explained previously, channel signals propagate from a desired source point to the transducer elements. The arrival time at the transducer elements may be a function of the distance from the source point to the transducer element and the sound speed of the medium through which the ultrasound waves travel, e.g., distance (source, transducer element)/medium sound speed, in the case in which the sound speed in the medium is a constant. When the sound speed in the medium is not constant, the arrival time at the transducer elements may be an integral over the incremental propagation times along the assumed propagation path, where the incremental propagation times are the incremental distances along the path divided by the sound speeds at each incremental distance. Thus, beamforming is performed to remove the assumed differences in arrival time at the transducer elements, typically using a single assumed sound speed for the medium, then the time-delayed signals are summed to form the image (explained below). If the assumed beamforming sound speed differs from the true sound speed in the medium, the beamforming will be sub-optimal and less than optimal image resolution and contrast will result. Thus, as explained herein, beamforming may be performed on the same channel signals with different sound speeds to identify the target or optimal sound speed for forming the image, based on a beamforming quality metric.

At 706, method 700 includes calculating a beamforming quality metric from the delayed channel data. The beamforming quality metric may be based on a coherence factor that represents a level of similarity among the delayed channel signals, which is correlated with image resolution and contrast but does not require an image to be formed. Additional details regarding the calculation of the beamforming quality metric are provided in more detail below with respect to FIG. 8. In some embodiments of method 700, at 707, method 700 includes calculating a beamforming quality metric over all channel data acquired at 702 and delayed at 704. Calculating over all the channel data may give a single beamforming quality metric associated with all data acquired to form an ultrasound image. In some embodiments of method 700, at 708, method 700 includes calculating a beamforming quality metric over subsets of the channel data, such that a beamforming quality metric is calculated for each of a plurality of scanning regions, as shown by the plurality of graphs 500 in FIG. 5. The regions may be formed by subdividing the entire area that is imaged into a default pattern of regions by the ultrasound imaging system, or an operator may select the pattern of regions. Calculating over subsets of the channel data may result in a beamforming quality metric being calculated for each region of the ultrasound scan.

At 709, method 700 includes repeating 702-706 for each different sound speed of the set of possible sound speeds. In this way, a beamforming quality metric is calculated for each sound speed of the set of possible sound speeds (whether for all the channel data or each subset of channel data).

At 710, method 700 includes calculating and fitting each beamforming quality metric as a function of the corresponding beamforming sound speed. This function may be represented as a graph (such as graph 302 of FIG. 3) with the beamforming sound speeds plotted on the x-axis of the graph and the beamforming quality metrics plotted on the y-axis of the graph. Each beamforming quality metric resulting from a sound speed may be plotted as a data point with a first coordinate represented by the beamforming sound speed and a second coordinate represented by the beamforming quality metric. In some embodiments of method 700, at 711, method 700 includes fitting all beamforming quality metrics calculated for all channel data. In this example, an ultrasound scan may include one region, which would be an entire anatomical region operated over by the ultrasound scan. All beamforming quality metric data as a result of different sound speeds may apply to the entire anatomical region operated over by the ultrasound scan, resulting in all data points being fitted as a function of beamforming sound speed. In some embodiments of method 700, at 712, method 700 includes fitting the beamforming quality metrics for each subset of channel data. In this example, an ultrasound scan may include a plurality of regions, and beamforming quality metrics may be fitted as a function of beamforming sound speed for each region in the ultrasound scan based on the channel data for each region.

At 713, method 700 includes calculating a target sound speed from the fit of the beamforming quality metric data. In one example, the curve fitted to the data may be a low-order polynomial, such as a degree-four polynomial. The polynomial fit may be represented as a best fit polynomial curve over the plotted data points on the graph, for a graph associated with an entire scanning region or each graph associated with a respective subset of an entire scanning region. The target sound speed may be identified as the sound speed where the polynomial curve fit for the beamforming quality metric obtains its maximum value. A range of uncertainty may also be calculated where a range of beamforming sound speeds have beamforming quality metrics smaller than, but within a tolerance value of, the largest beamforming quality metric in the polynomial fit, where the tolerance value in one example is based on the standard deviation of the polynomial fit from the calculated beamforming quality metric data. The range of uncertainty may represent a range of values based on a confidence that generating an ultrasound image using sound speeds in the range may generate an ultrasound image with optimal beamforming quality metrics. When the channel data is divided into regions, a target beamforming sound speed and a range of uncertainty may be identified for each region.

At 714, method 700 includes generating an ultrasound image or ultrasound images using time delays based on the target sound speed (or target sound speeds when a target sound speed is identified for each region). In one example, an ultrasound image may be generated using receive beamforming time delays or transmit beamforming time delays or both transmit and receive beamforming time delays calculated with the target sound speed(s). In another example, an ultrasound image may be generated using time delays calculated with a sound speed of a data point closest to the target sound speed. To generate the ultrasound image, the time delays are applied to the original (e.g., undelayed) receive channel data based on the selected sound speed(s), the time-delayed receive channel signals are summed to form a beamsum signal, a logarithm of an absolute value of the beamsum signal is determined, a scanconversion to square pixels is performed, and the pixels are scaled to 8-bit grayscale values to form the image. Method 700 then returns.

While method 700 is described herein as being applied on a single, original set of ultrasound receive channel signals (resulting from one set of transmits), in some examples, a new set of ultrasound receive channel signals may be obtained for each different sound speed. When only a single, original set of ultrasound receive channel signals is acquired, one transmit time delay may be applied for the transmit beamforming, which may be the nominal/assumed sound speed as described above. When multiple sets of ultrasound receive channel signals are acquired, the single transmit time delay may be applied for transmit beamforming for each set that is acquired, or different transmit time delays (e.g., matching the receive time delays) may be applied for each set.

FIG. 8 shows a flow chart illustrating an example method 800 for calculating a beamforming quality metric. Method 800 may be carried out according to instructions stored in non-transitory memory of a computing device, such as memory 120, and executed by a processor of the computing device, such as processor 116. In some examples, method 800 may be carried out as part of method 700, e.g., in order to calculate the beamforming quality metrics in order to determine the target sound speed for receive beamforming, transmit beamforming, or both transmit and receive beamforming.

At 802, method 800 includes acquiring channel signals for a set of ranges and a set of transverse directions (where the transverse directions are each transverse to a respective range direction). The receive channels 234 of FIG. 2 may be used to acquire the channel signals. These channel signals are defined for each element, range, and transverse direction such that sᵢ(r, θ), where i is a channel index, r is a range index, and B is a transverse direction index.

At 804, method 800 includes applying receive beamforming time delays to the acquired channel signals. Receive beamforming time delays may be applied to the acquired channel signals based on a selected sound speed, as described above with respect to FIG. 7.

At 806, method 800 includes calculating a coherence factor from the time delayed channel signals. The coherence factor is a value defined at each range and transverse direction, i.e., C(r, θ). The coherence factor may be defined (at least in one example) to be a ratio of sums of the channel signals. The coherence factor may be calculated according to the equation

$$C = \frac{\vert \sum {s}_{i} \vert}{\sum \vert {s}_{i} \vert}$$

such that the coherence factor is the ratio of two quantities: an amplitude of a sum of the time delayed channel signals (the absolute value of the coherent sum A of FIG. 2) and a sum of the amplitudes of the time delayed channel signals (the incoherent sum B of FIG. 2). The beamforming quality metric may be a normalized value in an inclusive range from 0 to 1, where 1 means high confidence that the magnitude of the beamsum represents the scattering from the intended focus direction and values closer to 0 may indicate that spurious and/or off-axis scattering may be present in the receive channel data. The ratio may be calculated in detection processor 252 of FIG. 2. However, other mechanisms for calculating the coherence factor may be utilized, such as a ratio of the squared amplitude of the channel signals, a similarity of the phases of complex channel signals, or a similarity of the signs of real channel signals.

At 808, method 800 includes calculating a trimmed mean of the coherence factor over a set of ranges and transverse directions. Calculating the trimmed mean may include excluding a percentage of the smallest values in the dataset before calculating a mean of the remaining values in the dataset. Calculating the trimmed mean may include calculating the cumulative distribution of the coherence factor values over a set of ranges and transverse directions; finding, from the cumulative distribution, a value Cₘᵢₙ such that a fraction p of the coherence factor values are smaller than Cₘᵢₙ; and calculating the mean of those coherence factor values that are larger than Cₘᵢₙ to produce the trimmed mean of the coherence factor. This trimmed mean is a single value defined for a set of ranges and transverse directions. In one example, p may be 0.75, but other values are possible without departing from the scope of this disclosure. However, in other examples, Cₘᵢₙ may be a predetermined value that is determined empirically rather than based on the cumulative distribution. In still further examples, a mean of all of the values may be determined. The coherence factor is a measure of the similarity of the time delayed channel signals. When the channel signals predominantly consist of echoes from the focus point, the similarity of the channel signals is high, and it is highest when the signals have been delayed with the correct receive beamforming time-delays. When the channel signals predominantly consist of echoes originating away from the focus point, however, the similarity of the channel signals is low, and it is lowest when the signals have been properly delayed. Thus, as the beamforming sound speed changes from a sub-optimum to an optimum value, the coherence factor will increase for some ranges and directions and will decrease for other ranges and directions. When averaged over samples with low and high coherence factors, the coherence factor is typically largest when the beamforming time delays for the optimum beamforming sound speed are applied. By excluding those samples with low coherence, the sensitivity of the mean coherence to changes in the beamforming sound speed is increased.

At 810, method 800 includes calculating a filtered derivative magnitude of the coherence factor in the direction transverse to the range direction. For a planar scan using a one-dimensional probe, there is one direction, the azimuthal direction, in the scan plane transverse to the range direction. For a volume scan using a two-dimensional matrix probe, there are two directions, azimuthal and elevational, in the volume scan transverse to the range direction. A beamforming point-spread-function may degrade more quickly in the transverse directions with arrival time errors than in the range direction, since the desired decrease in response in the transverse directions relies on cancellation of signals. The derivative of the coherence in a transverse direction is a measure of the width of the receive beamforming response profile, that is, the sharpness with which transverse edge-like features are reflected in the image. For a two-dimensional ultrasound scan format, only the azimuthal transverse dimension is present in the scan data, so only a derivative in the azimuthal direction can be calculated. For a volume ultrasound scan format, both an azimuthal and elevational dimension is present in the scan data, so that derivatives in both dimensions can be calculated. However, the derivative has a property of emphasizing high frequencies, so a low-pass filtered derivative is used to weight features in the data associated with beamforming performance and to mitigate noise amplification. The filtered derivative magnitude may be calculated by calculating a lowpass filter with a characteristic length L, where L is approximately the average correlation length of the coherence factor in the transverse direction. In one example, the lowpass filter may be a Gaussian filter with a fullwidth at half-maximum (FWHM) of L. The lowpass filter is convolved with the discrete derivative filter [1, −1] to produce a filtered derivative filter. The filtered derivative filter is applied in the transverse direction or directions to the coherence factor at a set of ranges, and the absolute value of the filtered derivative filter output is calculated to produce a filtered derivative magnitude of the coherence factor. This filtered derivative magnitude is a value defined at each range and transverse direction.

At 812, method 800 includes calculating a trimmed mean of the filtered derivative magnitude of the coherence factor. The trimmed mean of the filtered derivative magnitude of the coherence factor is calculated by calculating the cumulative distribution of the filtered derivative magnitude of the coherence factor over a set of ranges and transverse directions, finding a value Dₘᵢₙ such that a fraction q of the filtered derivative magnitude values are smaller than Dₘᵢₙ(with q=0.75 in one example), and calculating the mean of those filtered derivative magnitude values that are larger than Dₘᵢₙ to produce the trimmed mean of the filtered derivative magnitude values. Restricting the mean to those samples with the largest derivative magnitude values more heavily weights data values corresponding to edges in the tissue, which increases the sensitivity of the mean filtered derivative magnitude to changes in the beamforming sound speed. The trimmed mean is a single value defined for a set of ranges and transverse directions. However, in other examples, Dₘᵢₙ may be a predetermined value that is determined empirically rather than based on the cumulative distribution. In still further examples, a mean of all of the values may be determined.

At 814, method 800 includes calculating a beamforming quality metric based on the trimmed mean of the coherence factor and the trimmed mean of the filtered derivative magnitude of the coherence factor. The beamforming quality metric may be calculated by multiplying the trimmed mean of the coherence factor by the trimmed mean of the filtered derivative magnitude of the coherence factor. The beamforming quality metric is a single value defined for a set of ranges and transverse directions. Method 800 then returns.

Thus, methods 700 and 800 may be executed to determine a target sound speed for receive beamforming, for transmit beamforming, or for both transmit and receive beamforming, for generating an image. The same processing steps may be applied to the magnitude of the beamsum data or the log-scaled magnitude of the beamsum data rather than to the coherence factor. But in practice, using the coherence factor has generally shown to produce a more distinct change in the beamforming quality metric, and one more reliably correlated with image quality, because the coherence factor is a normalized quantity, limited to the range [0, 1], unlike the magnitude, or the log-scaled magnitude, of the beamsum.

To select the target beamforming sound speed, the beamforming quality metric may be calculated using a set of beamforming sound speeds cᵢ over a range of likely values. As explained above, undelayed channel signals may be acquired and stored and the respective beamforming quality metrics may be calculated using each of the beamforming sound speeds. Alternatively, the beamforming quality metrics may be calculated using different channel signals acquired with different beamforming sound speed time delays while scanning substantially the same tissue.

A low-order polynomial is then fit in the least-squares sense to the set of calculated beamforming quality metric values, Qᵢ. In an example, a polynomial of degree 4 is fit. It is numerically desirable to normalize the set of sound speed values to the range −1 to 1, using the transformation xᵢ=(2 cᵢ-cₘₐₓcₘᵢₙ)/(cₘₐₓ-cₘᵢₙ), where cᵢ is one of the sound speed values in the set, cₘₐₓ is the large sound speed value in the set, and cₘᵢₙ is the smallest sound speed value in the set. The least-squares fit then finds the coefficients a, b, c, d and e of the degree-4 polynomial, f(x)=ax⁴+bx³+cx²+dx+e, that minimizes the difference between the fit and the quality metric values, Σ|fᵢ-Qᵢ|².

The maximum of the fitted polynomial may either be calculated analytically or numerically. The value xₘₐₓ at which the polynomial reaches its maximum value corresponds to an optimal sound speed, cₒₚₜ=(cₘₐₓ+cₘᵢₙ)/2+xₘₐₓ(cₘₐₓ-cₘᵢₙ)/2.

Typically, the polynomial fit will not match the quality metric values Qᵢ at the set of sound speeds cᵢ. The mean-squared deviation of the fit, σ=[Σ|fᵢ-Qᵢ|²]^{1/2} may be used to estimate a range of plausible values of the optimal sound speed. The lower limit of the likely best normalized sound speed may be chosen as the value x₀ less than xₘₐₓ such that f(xₘₐₓ)-f(x₀)=σ. Similarly, the upper limit of the likely best normalized sound speed may be chosen as the value x₁ larger than xₘₐₓ such that f(xₘₐₓ)-f(x₁)=σ. These define the range of normalized sound speeds for which the fit deviates from its maximum value by σ, a measure of the typical deviation from the fit.

A technical effect of using a calculated beamforming quality metric to automatically determine an optimal beamforming sound speed parameter for ultrasound image generation is that ultrasound image quality may be improved without operator involvement. Manufacturers of ultrasound systems have found that most operators rarely use features that require manual intervention, such as turning knobs or pressing buttons, even if such features would result in better image quality. Another technical effect is that adaptive beamforming algorithms may use the calculated beamforming quality metric to choose time delays for a given scanning region, which may minimize image artifacts typically due to less than optimal time delay estimates. The beamforming quality metric can also be used in conjunction with other image improvement algorithms, such as adaptive beamforming, to quantify the degree of image improvement. If, for example, the image quality metric in one tissue region indicates that an adaptive beamforming algorithm has unintentionally degraded the image, then the adaptive beamforming algorithm could be suppressed for that tissue region. In addition, the beamforming quality metric can be used to automatically choose imaging parameters, such as the number of transmit focal zones, the number of transmit directions, the transmit f-number, transmit and receive apodization functions, as one input to a cost function to optimally balance image quality with other parameters of interest, such as frame rate and field of view. The beamforming quality metric can also be used to automatically choose fixed parameters, such as filter lengths or threshold values, for algorithms designed to improve image quality, thereby avoiding time-consuming testing and experimentation to choose those parameters.

The disclosure also provides support for a method, comprising: calculating a respective beamforming quality metric for each of a plurality of beamforming sound speeds, each beamforming quality metric calculated on ultrasound receive channel signals time-delayed based on a respective beamforming sound speed, identifying a target beamforming sound speed based on each beamforming quality metric, and generating an ultrasound image using the target beamforming sound speed. In a first example of the method, the method further comprises: acquiring and storing in memory the ultrasound receive channel signals and time-delaying the ultrasound receive channel signals multiple times, each time using a respective different beamforming sound speed. In a second example of the method, optionally including the first example, each respective different beamforming sound speed is selected from a set of possible beamforming sound speeds, such that a beamforming quality metric is calculated for each beamforming sound speed in the set of possible beamforming sound speeds. In a third example of the method, optionally including one or both of the first and second examples, generating the ultrasound image using the target beamforming sound speed comprises time-delaying the ultrasound receive channel signals based on the target beamforming sound speed. In a fourth example of the method, optionally including one or more or each of the first through third examples, generating the ultrasound image using the target beamforming sound speed comprises time-delaying ultrasound signals output by each ultrasound transducer of an ultrasound probe based on the target beamforming sound speed. In a fifth example of the method, optionally including one or more or each of the first through fourth examples, identifying the target beamforming sound speed based on each beamforming quality metrics comprise fitting respective beamforming quality metrics for each corresponding beamforming sound speed to a function of a beamforming sound speed, and identifying the target beamforming sound speed from the fitted function. In a sixth example of the method, optionally including one or more or each of the first through fifth examples, identifying the target beamforming sound speed from the fitted function comprises identifying the target beamforming sound speed as a beamforming sound speed that results in a beamforming quality metric that is closest to a largest beamforming quality metric of the fitted function. In a seventh example of the method, optionally including one or more or each of the first through sixth examples, identifying the target beamforming sound speed from the fitted function comprises identifying the target beamforming sound speed as a beamforming sound speed that results in a largest beamforming quality metric of the fitted function. In an eighth example of the method, optionally including one or more or each of the first through seventh examples, calculating the respective beamforming quality metric comprises, for a first beamforming quality metric calculated for the ultrasound receive channel signals time-delayed based on a first beamforming sound speed: calculating a first coherence factor for each range and transverse direction from the ultrasound receive channel signals, calculating a first trimmed mean over each range and transverse direction from each first coherence factor, calculating a filtered derivative magnitude of each first coherence factor in each transverse direction, calculating a second trimmed mean over each range and transverse direction from each filtered derivative magnitude, and calculating the first beamforming quality metric by multiplying the first trimmed mean by the second trimmed mean. In a ninth example of the method, optionally including one or more or each of the first through eighth examples, the ultrasound receive channel signals are received over a plurality of channels, each channel coupled to a respective ultrasound transducer of an ultrasound probe, and wherein each first coherence factor is calculated as a ratio of an absolute value of summed output of each channel to a sum of absolute values of output of each channel.

The disclosure also provides support for a system, comprising: a plurality of ultrasound transducers configured to transmit and receive ultrasound signals, a memory storing instructions, and a processor configured to execute the instructions to: acquire, via the plurality of ultrasound transducers, a set of ultrasound receive channel signals, calculate a respective beamforming quality metric for each of a plurality of time-delayed sets ultrasound receive channel signals, where each time-delayed set of ultrasound receive channel signals is time-delayed from the set of ultrasound receive channel signals based on a different beamforming sound speed, identify a target beamforming sound speed based on each beamforming quality metric, and generate an ultrasound image using the target beamforming sound speed. In a first example of the system, the set of ultrasound receive channel signals is received via a set of receive channels each coupled to an ultrasound transducer of the plurality of ultrasound transducers, and wherein each beamforming quality metric is calculated from a coherence factor that is a measure of similarity among channel signals of each time-delayed set of ultrasound receive channel signals. In a second example of the system, optionally including the first example, a first coherence factor is calculated for each range and transverse direction from a first time-delayed set of ultrasound receive channel signals time-delayed by a first beamforming sound speed, a first trimmed mean is calculated over each range and transverse direction from each first coherence factor, a filtered derivative magnitude of each first coherence factor is calculated in each transverse direction, a second trimmed mean over each range and transverse direction is calculated from each filtered derivative magnitude, and a first beamforming quality metric is calculated by multiplying the first trimmed mean by the second trimmed mean. In a third example of the system, optionally including one or both of the first and second examples, identifying the target beamforming sound speed based on the beamforming quality metrics comprises fitting respective beamforming quality metrics for each corresponding beamforming sound speed to a function of a beamforming sound speed, and identifying the target beamforming sound speed from the fitted function. In a fourth example of the system, optionally including one or more or each of the first through third examples, the processor is configured to execute the instructions to calculate each beamforming quality metric before the ultrasound image is generated, and wherein generating the ultrasound image using the target beamforming sound speed comprises applying the target beamforming sound speed to calculate transmit beamforming time delays, receive beamforming time delays, or both transmit and receive beamforming time delays.

The disclosure also provides support for a method, comprising, generating an ultrasound image from a plurality of receive channel signals, including, for each region of a plurality of regions of the ultrasound image, time-delaying a set of the plurality of receive channel signals corresponding to that region based on a respective beamforming sound speed, where each beamforming sound speed is selected for each region independently based on a set of beamforming quality metrics calculated for each region. In a first example of the method, the plurality of regions of the ultrasound image includes a first region generated from a first set of the plurality of receive channel signals and a second region generated from a second set of the plurality of receive channel signals, wherein the first set of the plurality of receive channel signals is time-delayed based on a first beamforming sound speed and the second set of the plurality of receive channel signals is time-delayed based on a second beamforming sound speed, different than the first beamforming sound speed. In a second example of the method, optionally including the first example, the method further comprises: selecting the first beamforming sound speed by calculating a first set of beamforming quality metrics for the first region, the first set of beamforming quality metrics calculated by: time-delaying the first set of the plurality of receive channel signals multiple times, each based on a different beamforming sound speed selected from a set of beamforming sound speeds, and calculating a beamforming quality metric each time. In a third example of the method, optionally including one or both of the first and second examples, calculating the beamforming quality metric each time comprises calculating a coherence ratio for each range and transverse direction of the first set of the plurality of receive channel signals and determining a mean coherence ratio, each coherence ratio comprising an absolute value of summed time-delayed channel signals to a sum of absolute values of time-delayed channel signals. In a fourth example of the method, optionally including one or more or each of the first through third examples, selecting the first beamforming sound speed comprises fitting the first set of beamforming quality metrics as a function of a corresponding sound speed applied to calculate each beamforming quality metric and selecting the first beamforming sound speed from the fit.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

1. A method, comprising:
calculating a respective beamforming quality metric for each of a plurality of beamforming sound speeds, each beamforming quality metric calculated on ultrasound receive channel signals time-delayed based on a respective beamforming sound speed;
identifying a target beamforming sound speed based on each beamforming quality metric; and
generating an ultrasound image using the target beamforming sound speed.

2. The method of claim 1, further comprising acquiring and storing in memory the ultrasound receive channel signals and time-delaying the ultrasound receive channel signals multiple times, each time using a respective different beamforming sound speed.

3. The method of claim 2, wherein each respective different beamforming sound speed is selected from a set of possible beamforming sound speeds, such that a beamforming quality metric is calculated for each beamforming sound speed in the set of possible beamforming sound speeds.

4. The method of claim 2, wherein generating the ultrasound image using the target beamforming sound speed comprises time-delaying the ultrasound receive channel signals based on the target beamforming sound speed.

5. The method of claim 2, wherein generating the ultrasound image using the target beamforming sound speed comprises time-delaying ultrasound signals output by each ultrasound transducer of an ultrasound probe based on the target beamforming sound speed.

6. The method of claim 1, wherein identifying the target beamforming sound speed based on each beamforming quality metric comprises fitting respective beamforming quality metrics for each corresponding beamforming sound speed to a function of a beamforming sound speed, and identifying the target beamforming sound speed from the fitted function.

7. The method of claim 6, wherein identifying the target beamforming sound speed from the fitted function comprises identifying the target beamforming sound speed as a beamforming sound speed that results in a beamforming quality metric that is closest to a largest beamforming quality metric of the fitted function.

8. The method of claim 6, wherein identifying the target beamforming sound speed from the fitted function comprises identifying the target beamforming sound speed as a beamforming sound speed that results in a largest beamforming quality metric of the fitted function.

9. The method of claim 1, wherein calculating the respective beamforming quality metric comprises, for a first beamforming quality metric calculated for the ultrasound receive channel signals time-delayed based on a first beamforming sound speed:
calculating a first coherence factor for each range and transverse direction from the ultrasound receive channel signals;
calculating a first trimmed mean over each range and transverse direction from each first coherence factor;
calculating a filtered derivative magnitude of each first coherence factor in each transverse direction;
calculating a second trimmed mean over each range and transverse direction from each filtered derivative magnitude; and
calculating the first beamforming quality metric by multiplying the first trimmed mean by the second trimmed mean.

10. The method of claim 9, wherein the ultrasound receive channel signals are received over a plurality of channels, each channel coupled to a respective ultrasound transducer of an ultrasound probe, and wherein each first coherence factor is calculated as a ratio of an absolute value of summed output of each channel to a sum of absolute values of output of each channel.

11. A system, comprising:
a plurality of ultrasound transducers configured to transmit and receive ultrasound signals;
a memory storing instructions; and
a processor configured to execute the instructions to:
acquire, via the plurality of ultrasound transducers, a set of ultrasound receive channel signals;
calculate a respective beamforming quality metric for each of a plurality of time-delayed sets ultrasound receive channel signals, where each time-delayed set of ultrasound receive channel signals is time-delayed from the set of ultrasound receive channel signals based on a different beamforming sound speed;
identify a target beamforming sound speed based on each beamforming quality metric; and
generate an ultrasound image using the target beamforming sound speed.

12. The system of claim 11, wherein the set of ultrasound receive channel signals is received via a set of receive channels each coupled to an ultrasound transducer of the plurality of ultrasound transducers, and wherein each beamforming quality metric is calculated from a coherence factor that is a measure of similarity among channel signals of each time-delayed set of ultrasound receive channel signals.

13. The system of claim 12, wherein:
a first coherence factor is calculated for each range and transverse direction from a first time-delayed set of ultrasound receive channel signals time-delayed by a first beamforming sound speed;
a first trimmed mean is calculated over each range and transverse direction from each first coherence factor;
a filtered derivative magnitude of each first coherence factor is calculated in each transverse direction;
a second trimmed mean over each range and transverse direction is calculated from each filtered derivative magnitude; and
a first beamforming quality metric is calculated by multiplying the first trimmed mean by the second trimmed mean.

14. The system of claim 11, wherein identifying the target beamforming sound speed based on each beamforming quality metric comprises fitting respective beamforming quality metrics for each corresponding beamforming sound speed to a function of a beamforming sound speed, and identifying the target beamforming sound speed from the fitted function.

15. The system of claim 11, wherein the processor is configured to execute the instructions to calculate each beamforming quality metric before the ultrasound image is generated, and wherein generating the ultrasound image using the target beamforming sound speed comprises applying the target beamforming sound speed to calculate transmit beamforming time delays, receive beamforming time delays, or both transmit and receive beamforming time delays.

16. A method, comprising;
generating an ultrasound image from a plurality of receive channel signals, including, for each region of a plurality of regions of the ultrasound image, time-delaying a set of the plurality of receive channel signals corresponding to that region based on a respective beamforming sound speed, where each beamforming sound speed is selected for each region independently based on a set of beamforming quality metrics calculated for each region.

17. The method of claim 16, wherein the plurality of regions of the ultrasound image includes a first region generated from a first set of the plurality of receive channel signals and a second region generated from a second set of the plurality of receive channel signals, wherein the first set of the plurality of receive channel signals is time-delayed based on a first beamforming sound speed and the second set of the plurality of receive channel signals is time-delayed based on a second beamforming sound speed, different than the first beamforming sound speed.

18. The method of claim 17, further comprising selecting the first beamforming sound speed by calculating a first set of beamforming quality metrics for the first region, the first set of beamforming quality metrics calculated by:
time-delaying the first set of the plurality of receive channel signals multiple times, each based on a different beamforming sound speed selected from a set of beamforming sound speeds, and calculating a beamforming quality metric each time.

19. The method of claim 18, wherein calculating the beamforming quality metric each time comprises calculating a coherence ratio for each range and transverse direction of the first set of the plurality of receive channel signals and determining a mean coherence ratio, each coherence ratio comprising an absolute value of summed time-delayed channel signals to a sum of absolute values of time-delayed channel signals.

20. The method of claim 18, wherein selecting the first beamforming sound speed comprises fitting the first set of beamforming quality metrics as a function of a corresponding sound speed applied to calculate each beamforming quality metric and selecting the first beamforming sound speed from the fit.
